(19) United States
(12) Patent Application Publication
(10) Pub. No.: US 2001/0021499 A1
(43) Pub. Date: Sep. 13, 2001

(54) Method for preparing a model system for cellular insulin resistance and device for use with the model system

(76) Inventors: Antek Wielburski, Stockholm (SE); Harriet Ronnholm, Trangsund (SE)

Correspondence Address: DINSMORE & SHOHL, LLP, 1900 CHEMED CENTER, 255 EAST FIFTH STREET, CINCINNATI, OH, 45202, US

(21) Appl. No.: 09/753,212

(22) Filed: Jan. 2, 2001

Related U.S. Application Data
(60) Provisional application No. 60/177,469, filed on Jan. 21, 2000.

(30) Foreign Application Priority Data
Dec. 30, 1999 (SE) 9904849-8

(51) Int. Cl. C12Q 1/00; C12N 5/06

(52) U.S. Cl. 435/4; 435/325

(57) ABSTRACT

A method of preparing a cellular in vitro model system for insulin resistance by inducing insulin resistance to an animal cell culture in a cell culture medium comprises incubating the cell culture in the presence of glucose and at least one fatty acid, preferably a long-chain fatty acid, wherein the concentration of glucose is in the range of about 5 to about 25 mM and the concentration of fatty acid is less than about 2 mM. A device that may be used in the method comprises a cell culture flask (1), and a support member (2) for a carbon dioxide absorbent body, which support member (2) is partially insertable into the culture flask (1) and fixable in the flask opening with the absorbent body extending into the flask.

### FIELD OF THE INVENTION

The present invention relates to the preparation of a novel insulin resistant cell based model system, to the use thereof, and to a method and device, respectively, for use in the model.

### BACKGROUND OF THE INVENTION

Insulin resistance is frequently found in obese subjects and is an early hallmark in subjects prone to develop non-insulin-dependent diabetes (NIDDM). It can be defined as a diminution of the biological response to a given concentration of insulin. There are a number of factors that have been demonstrated to accelerate the development of insulin resistance in vivo. The most important among these are elevated blood levels of glucose and circulating free fatty acids. One major difficulty in attempts to study insulin resistance is that there is not yet any quantitative definition available. What is even more important is the fact that very little is known about pathogenesis of insulin resistance on molecular basis. During the last decades a number of methods have been developed using cellular systems to study pre-diabetic or diabetic states. In most of the cases induction of insulin resistance was achieved by using supra non-physiological concentrations of glucose (>25 μm) or/and insulin in cell culture media. These simplified approaches had several drawbacks such as lack of reproducibility and limitation of achieved effects depending on the cell type studied.

Schmitz-Peiffer, C., et al. (1999) J. Biol. Chem. 274, 24202-24210 discloses incubation of myoblasts with free fatty acids (FFA) of the concentration 0.2 to 2 mM to provide a model of lipid-induced skeletal muscle insulin resistance.

The use of insulin resistance models often involves the measurement of glucose and fatty acid oxidation rates. Usually, this has required complicated apparatus, common experimental set-ups using cells in suspension which is not satisfactory as many of the studied cell types are of an adherent type.

Ross, Philip, D., et al. (1981) Anal. Biochem. 112(2), 378-86, discloses a radiospirometer for continuous quantitation of ¹⁴CO₂ release for specifically labeled substrates by intact cultured cells attached to plastic petri dishes. The petri dish is sealed with a cover, and a carrier gas is bubbled under the surface of the growth medium. Labeled CO₂ is removed from the carrier gas by trapping in an organic base and quantitated by liquid scintillation counting.

Mark van Epps-Fung et al. (1997) Endocrinology, Vol 188, Nr 10, 4338-4345, discloses incubation of adipocytes with 10 nM glucose and 1 mM fatty acid. Thus a very small amount of glucose was used for the measurement of glucose transport.

### SUMMARY OF THE INVENTION

It is an object of the present invention to prepare a model for insulin resistance that overcomes the drawbacks of the prior art models. According to the invention, it has surprisingly been found that an excellent in vitro cellular model for insulin resistance in an animal (including humans) may be induced by long term incubation (usually from about eight hours or longer) of cell cultures in a cell culture medium medium containing moderately elevated, compared with normo-physiological levels, concentrations of glucose and free fatty acid (FFA).

In one aspect, the present invention therefore provides a method of inducing insulin resistance to an animal (including human) cell culture in a cell culture medium, which method comprises incubating the cell culture in the presence of glucose and at least one fatty acid, preferably a long-chain fatty acid, wherein the concentration of glucose is in the range of about 5 to about 25 mM and the concentration of fatty acid is less than about 2 mM.

Preferably, the concentration of glucose is in the range of about 10 to 20 mM and the concentration of fatty acid is in the range of about 120 PM to about 2 mM.

Preferred fatty acids are palmitic acid, oleic acid and linoleic acid.

The most preferred fatty acid for use in the method is palmitic acid.

The method of the invention may be applied to a variety of cells systems, including all cells affected in diabetes and obesity status. Exemplary cell systems are skeletal muscle cells, insulin secreting cells (i-like cells), adipocytes and hepatocytes.

In a second aspect, the present invention provides the use of the method for drug/target related studies, including, for example, screening of insulin releasing, insulin sensitizing or insulin mimetic compounds, metabolic pathway analysis, differential display analysis, signaling pathway analysis etc.

A third aspect of the invention relates to a method and device, respectively, for measuring carbohydrate and fatty acid oxidation rates by cultured cells in vitro, which method and device may be used with the model system prepared by the method of the invention as well as with other systems.

### BRIEF DESCRIPTION OF THE DRAWINGS

FIG. 1A is a schematic sectional view of an illustrative device for the determination of glucose and fatty acid oxidation rates.

FIG. 1B is a schematic perspective view of the separate parts of a practical design of the device in FIG. 1A.

FIG. 2 is diagram showing the effects of increasing concentrations of glucose on insulin dependent glucose uptake.

FIG. 3 is a diagram showing the effect of increasing concentrations of palmitate in the presence of low glucose content (5.5 mM) on insulin stimulatable glucose uptake.

FIG. 4 is a diagram showing a comparison of glucose uptake rates under normal conditions versus insulin resistance induced conditions.

FIG. 5 is a diagram showing the effects of increasing concentration of palmitate on glucose oxidation rates.

FIG. 6 is a diagram showing the effects of increasing glucose concentrations on glucose oxidation rates, as well as the effect of the combination of different glucose concentrations with 480 μM palmitate on glucose oxidation rates.

FIG. 7 is a diagram showing an example of a practical application of established insulin resistant cell model in evaluation of effects of potential PPAR ligands on glucose uptake rates.

### DETAILED DESCRIPTION OF THE INVENTION

The present invention is based on the fact that concentrations of glucose and circulating free fatty acids in blood from diabetic and obese patients are elevated. As mentioned above, the invention resides in the provision of a cellular based model of insulin resistance obtained by incubation of cell cultures in media containing only moderately elevated concentrations of both glucose and fatty acid, such as palmitic acid, compared with normal physiological levels. Thus, while most of the prior art studies within this area utilized one of the potential factors at a time, and at rather extreme and acute hyperglycemic and/or hyperinsulinemic conditions, which might influence insulin action, the present invention instead combines the glucose and fatty acid parameters during cell culture cultivation to reflect a chronically pre-diabetic state which in the end leads to fully developed insulin resistance.

By monitoring a number of metabolic read outs such as glucose uptake, glucose oxidation and fatty acid oxidation rates in response to action of insulin, the model prepared according to the invention permits a number of applications within the drug/target hunting area, such as metabolic pathway analysis, differential display analysis, signaling pathway analysis, as well as for screening of insulin releasers, insulin sensitizers, insulin mimetics, etc.

The invention will now be described in more detail in the following non-limiting Example. While the Example below describes exclusively a skeletal muscle system, the invention can, of course, be applied to other cellular systems, including all cells affected in diabetes and obesity states, such as e.g. insulin secreting cells, adipocytes and hepatocytes. One and each of the named cell types has its own specificity in terms of its specialized functions which in turn serve as a specific read out (insulin secretion, triglyceride synthesis, glucose production).

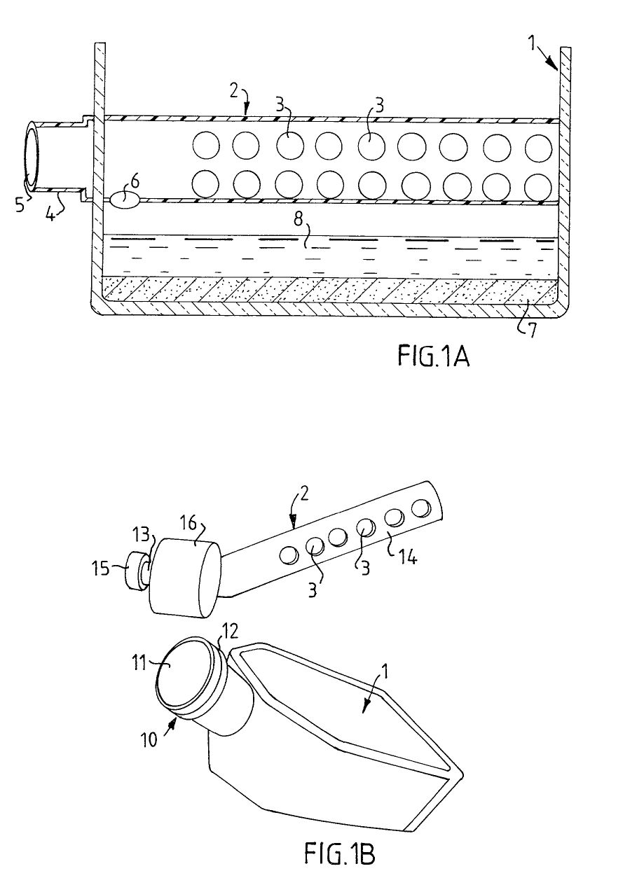

First, however, a device used in the Example will be described with reference to FIG. 1A.

The device comprises a cell culture flask, generally designated by reference numeral 1. Mounted in the flask 1 is a tube 2 having a plurality of holes or apertures 3 in the tubular wall and adapted to receive a rolled up (liquid-soaked) filter paper (not shown) in the apertured section thereof, such that the filter paper is in contact with the atmosphere within the flask through the apertures 3. The tube 2 has an end part 4 fitting through the flask opening and sealed by a septum 5. An aperture 6 made in the tube wall near the flask opening permits the needle of a syringe which has pierced the septum 5 to be inserted into the interior of the flask 1. In the illustrated case, the flask 1 contains a layer of adherent cells 7 and a culture medium 8.

The device may be used for measuring the cellular oxidation rates of substances, or substrates, where one of the final products is carbon dioxide. To that end a substrate labeled by a radioactive carbon isotope, such as ¹⁴C, is added to the flask containing adherent cells and culture medium. A filter paper soaked in a CO₂-trapping agent, e.g. hyamine solution (hyamine is a strong base), is rolled up and placed in the tube 2, and after a pre-determined incubation time, the incubation is stopped by adding e.g. sulfuric acid to the culture medium via a syringe, the needle piercing the septum 5 and extending through aperture 6. After additional incubation, the filter paper is removed, cut into pieces and transferred to a scintillation vial and the radioactivity is measured.

FIG. 1B illustrates a practical design of the device in FIG. 1A. Corresponding parts are designated by the same reference numerals as in FIG. 1A. The culture flask 1 is of standard type and has a tubular inlet part 10 with an opening 11 and an external thread 12. The support tube 2 for the filter paper, which tube is a separate part designed to be inserted into the flask 1, has a fore part 13 slightly angled to a rear part 14 provided with a number of holes 3 and adapted to receive the rolled up hyamine-soaked filter paper (not shown). The fore end of the tube 2 is sealed, 15. The insertable tube 2 is arranged to be inserted through the flask opening 11 and kept in position by a screw cap 16 (here shown on the tube 2) engaging with the thread 12 of the inlet part 10 and acting against an o-ring (not shown) which is secured on the tube 2 and abuts the edge of the flask opening 11 so that the system is closed.

### EXAMPLE

Materials

Rat L6 cells were obtained from The American Type Culture Collection (ATCC). Bovine insulin, Dulbecco's Modified Eagle's medium (DMEM), Phosphate Buffered Saline (PBS), Foetal Calf Serum (FCS), Penicillin and Streptomycin (PEST) were bought from Gibco Laboratories. Tissue culture plates were purchased from Costar. Bovine Serum Albumin (BSA) and cytochalasin B were obtained from Sigma, USA. U-¹⁴C-glucose, ³H-2-deoxy-glucose and U-¹⁴C-palmitate were from Du Pont NEN, Medical Scandinavia, Sweden. Whatman no. 1 filter paper was from Kebo Lab., Sweden, and Hyamine hydroxide from ICN, USA.

Methods

Cell Cultures

Rat L6 myoblasts were grown on culture flasks in Dulbecco's modified Eagle's medium (DMEM) containing 10% FCS and 2% PEST. To initiate differentiation, the media of sub-confluent cell cultures were replaced with DMEM supplemented with 1% FCS and 0.3 μM insulin as described in Klip, A., et al. (1984) Am. J. Physiol. 247, E291-E296; and Walker, P. S., et al. (1989) J. Biol. Chem. 264, 6587-6595.

Induction of Insulin Resistance

Differentiated skeletal muscle cells were incubated in serum free DMEM medium supplemented with 12 mM glucose and 480 μM palmitate bound to BSA in a molar ratio 5:1 for 20 hours in a standard cell culture incubator. For glucose uptake determinations the cells were seeded in 24-well plates and for substrate oxidation determinations cells were cultivated in T-25 flasks. One-hour prior to the measurement insulin was added at a concentration of 176 nmol/L.

Determination of Glucose Uptake Rate.

Glucose uptake was measured as described by Hundal H. S., Bilan P. J., Tsakiridis T., Marette A., Klip A. (1994.) Biochem. J., 297:289-295. Briefly, after incubation with hormones for 45 minutes, if not otherwise stated, cell monolayers were rinsed with glucose free PBS. Glucose uptake was quantified by incubating the cells in the presence of 1 μCi/ml ³H-2-deoxy-glucose in PBS for 8 min. Non-specific uptake was determined by quantifying cell-associated radioactivity in the presence of 10 μM cytochalasin B. Uptake of 2-deoxy-glucose was terminated by rapidly aspirating the medium, followed by three successive washes of cell monolayers with ice cold PBS. The cells were lysed in 0.5 M NaOH, followed by liquid scintillation counting. Rates of transport were normalized for protein content in each well.

Determination of Glucose and Palmitic Acid Oxidation Rates By ¹⁴CO₂ Trapping Method in Adherent Cells in Vitro.

In order to determine an efficiency by which glucose and free fatty acids (FFA) are converted into energy in cultured cells, a method for measuring rate of oxidative phosphorylation of these nutrients has been developed.

The principle of the glucose/FFA oxidation assay is based on the fact that one of the final products along metabolic pathways of these two substrates is carbon dioxide. Since the substrates are uniformly ¹⁴C labeled, the radioactivity of carbon dioxide trapped in a carbon dioxide trap is a direct measure of the metabolic activity in studied cells (Rodbell, M. (1964), J. Biol. Chem. 239, 375-380).

The cells were cultivated until sub-confluence in T-25 Costar flasks. Prior to the experiment, the cells were deprived of serum for 6 hours in DMEM medium containing 5 mM glucose. 3 ml of medium supplemented with (U-¹⁴C)-glucose or (U-¹⁴C)-palmitic acid (0.2 μCi/ml of each) were added to each flask. A filter paper (1.5×5.5 cm) soaked in hyamine solution was rolled up, blotted on a paper towel to remove excess of fluid, and placed carefully into the tube (2) of the device illustrated in Figures 1A and 1B and described above. The tube was mounted in the flasks, the screw caps (16) were tightened and cells were incubated for indicated time periods.

Incubation was stopped by carefully piercing the septum of the device with a 21 G needle attached to a 1 ml syringe containing 0.4 ml of 2 M sulfuric acid. The sulfuric acid was added into medium and the cells were incubated for additional 60 min. at 37° C. After this time interval, the filter paper was removed, cut into small pieces and transferred to scintillation vials containing 10 ml of scintillation solution. Methanol (0.2 ml) was added to each counting vial to increase the solubility of hyamine-CO₂ in the scintillation fluid. Finally the radioactivity was measured.

The remaining cells were washed briefly with ice cold PBS, solubilized with 1 M KOH and the protein content was determined according to the Bradford method (Bradford, M. M. 1976, Anal. Biochem. 72, 248-254).

Calculations

General.

The rate of substrate oxidation was obtained by correcting the observed number of disintegrations per minute for counting efficiency, milligram of protein in the culture flask, trapping interval, and a specific activity of the substrate at time zero using the following equation:

$$R = \frac{( D - B )}{S \times T \times M}$$

where

R=rate of substrate oxidation (μmol/min.×mg. prot.)

D=radioactivity on filter (dpm)

B=background (dpm)

S=specific radioactivity of substrate (dpm/μmol)

T=time (minutes)

M=protein content of the cultured cell plate/flask (mg. prot.)

Glucose Oxidation Rate;

Specific radioactivity was determined as follows. The radioactivity of a medium sample was measured (e.g. 100 μl gives approx. 40,000 dpm). Since the glucose concentration in medium was 5.5 mmol/l the specific radioactivity was calculated to 400,000 dpm/5.5 μmol (72,727 dpm/μmol).

Palmitic Acid Oxidation Rate;

Labeled palmitate added to the cultured cells was assumed to be the sole source of this substrate under the experimental conditions. For this reason the calculation of specific radioactivity differs from the above example. Specific radioactivity was determined by the manufacturer, in case of uniformly labeled palmitate it was 850 mCi/mmol. Since 0.2 μCi palmitate/ml medium are added, it was calculated that the palmitate concentration added is 0.2353 nmol/ml. Again, by measuring radioactivity of e.g. 100 μl medium the specific radioactivity expressed as dpm/nmol substrate was calculated.

Analyses

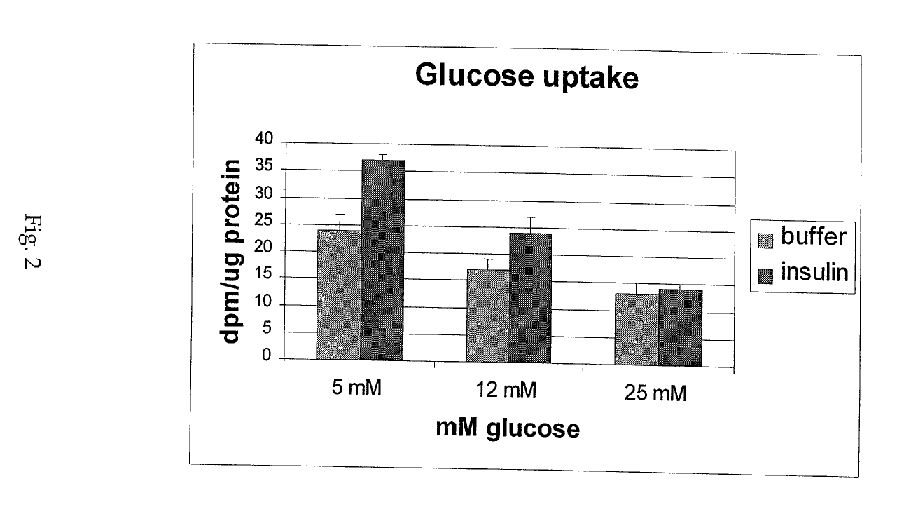

The effects of increasing concentrations of glucose on insulin dependent glucose uptake was studied with the model system described above, and the results are presented in FIG. 2. As can be seen in the figure, a maximal inhibitory effect is observed at a glucose concentration of 25 mM.

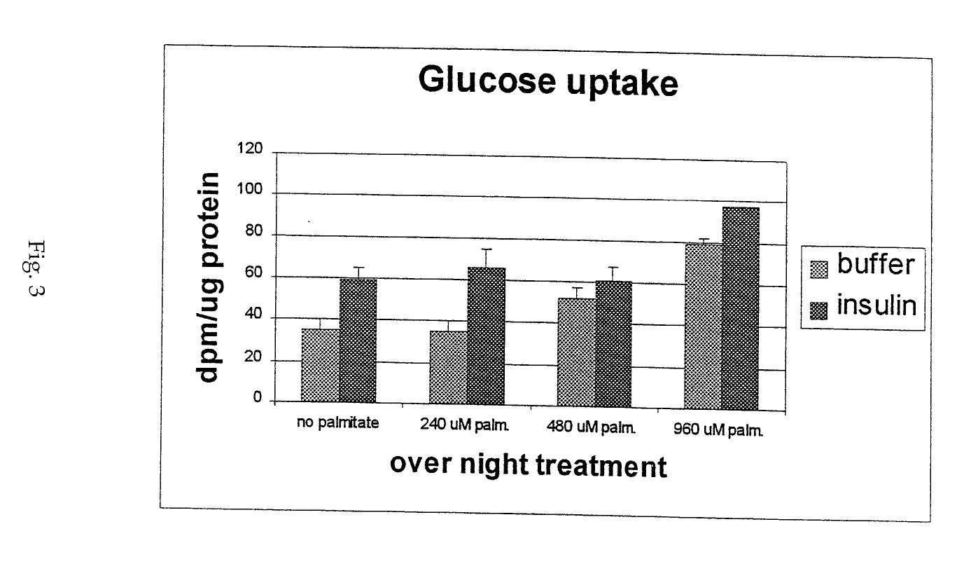

Also the effect of increasing concentrations of palmitate in the presence of low glucose content (5.5 mM) on insulin stimulatable glucose uptake was studied. The results are presented in FIG. 3. As shown, at the palmitate concentration of 480 μM the basal glucose uptake rate is slightly increased compared to control level, but the insulin effect is strongly inhibited.

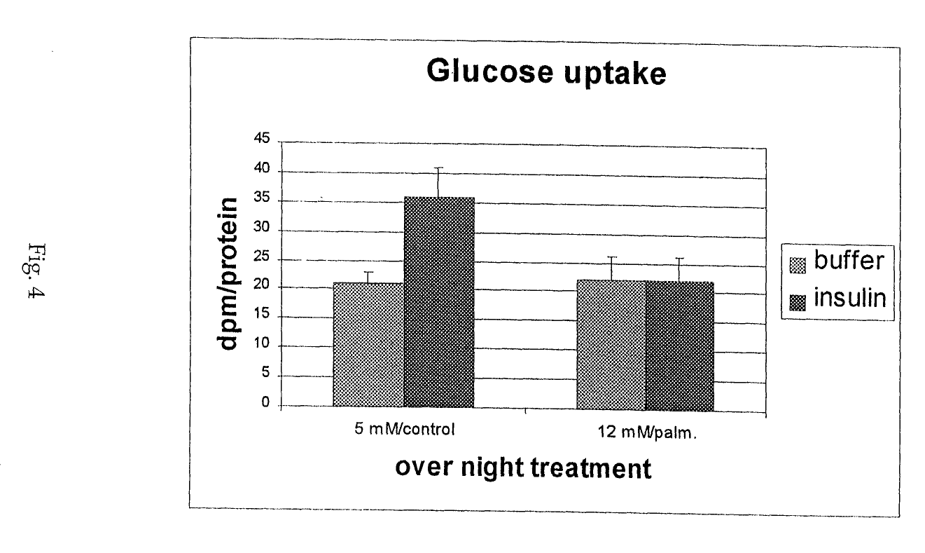

A comparison of glucose uptake rates under normal conditions versus insulin resistance induced conditions was made. The results are presented in FIG. 4. As can be seen in the figure, the basal glucose uptake rate is not affected by treatment of cell cultures with 12 mM glucose and 480 μM palmitate but the insulin effect is completely abolished.

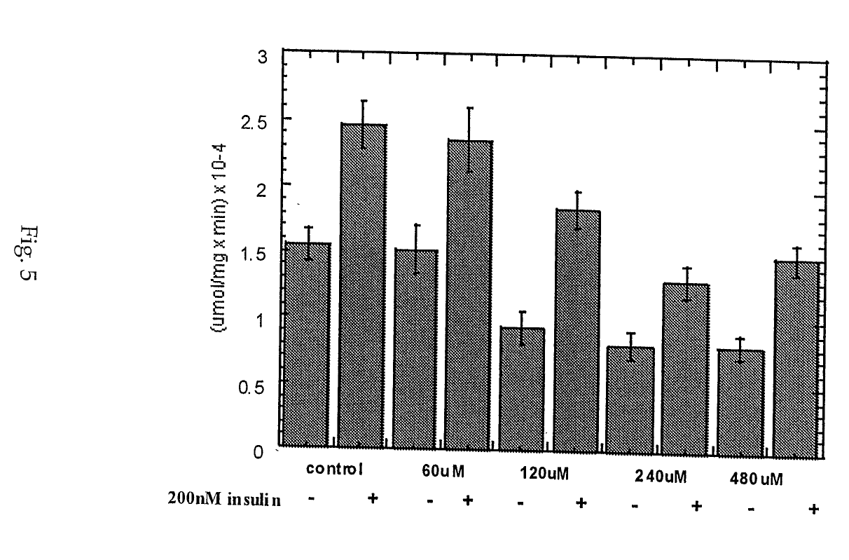

The effects of increasing concentrations of palmitate on glucose oxidation rates was also studied, and the results are illustrated in FIG. 5. Shown in the figure is a direct effect of increased palmitate concentration on a basal glucose oxidation rate as an effect of substrate preference. Also, an insulin dependent increase of glucose oxidation rates is decreased in a dose dependent mode. The glucose concentration was maintained at 5.5 mM throughout the experiment.

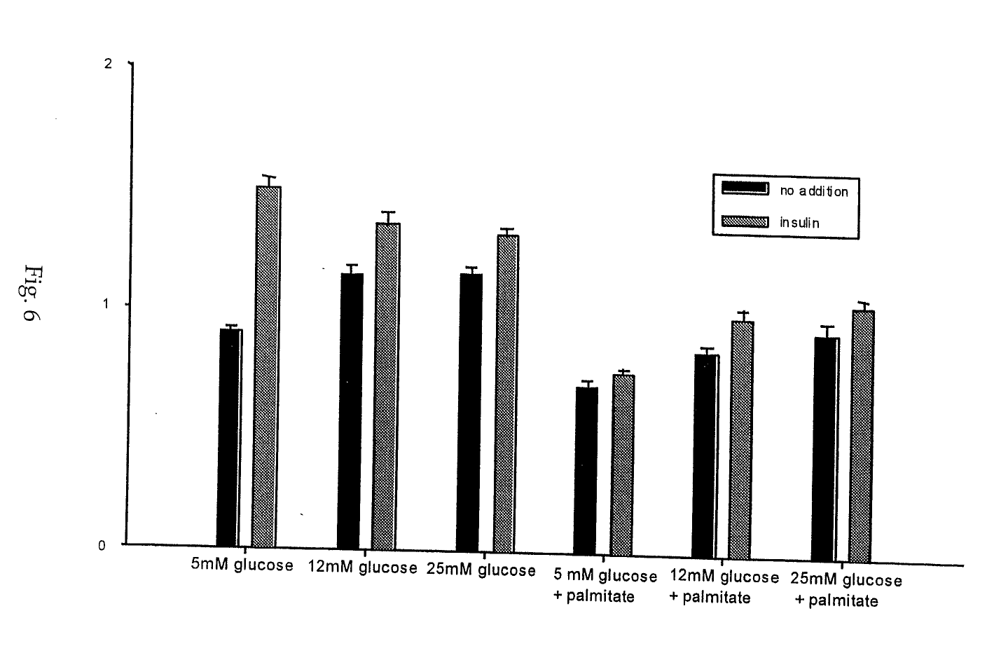

The results from a study of the effects of increasing glucose concentrations on the glucose oxidation rates are shown in FIG. 6. The figure also shows the effect of combination of different glucose concentrations with 480 μM palmitate on glucose oxidation rates.

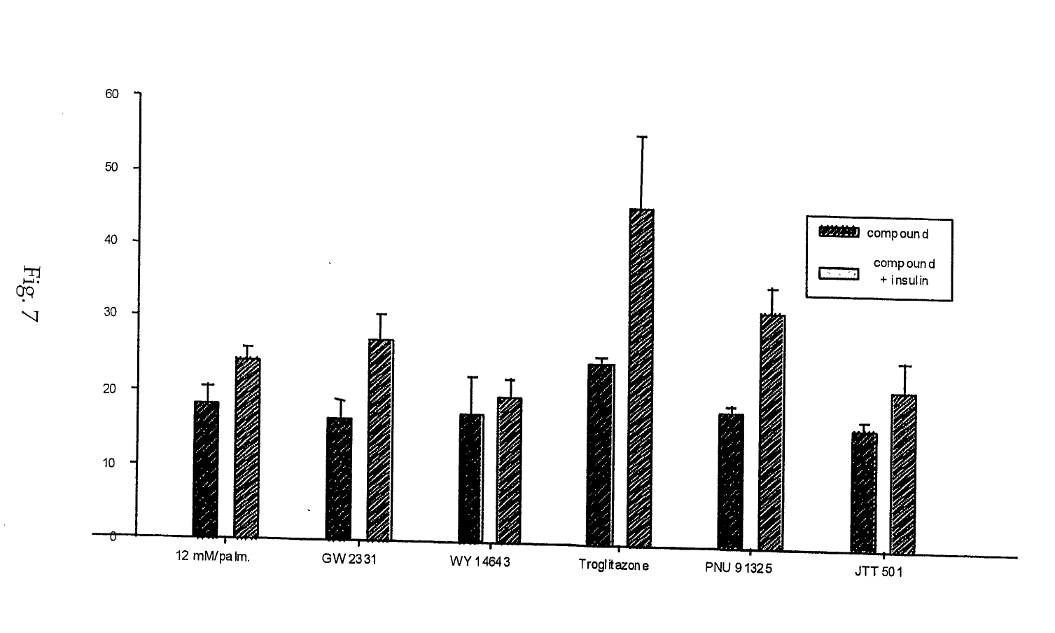

Finally, an example of a practical application of the established insulin resistant cell model in the evaluation of effects of potential PPAR ligands on glucose uptake rates is shown in FIG. 7.

1. A method of preparing a cellular in vitro model system for insulin resistance by inducing insulin resistance to an animal cell culture in a cell culture medium, which method comprises incubating the cell culture in the presence of glucose and at least one fatty acid, preferably a long-chain fatty acid, wherein the concentration of glucose is in the range of about 5 to about 25 mM, and the concentration of fatty acid is less than about 2 mM, preferably in the range of about 120 μM to about 2 mM.

2. The method according to claim 1, wherein the concentration of glucose is in the range of 10 to 20 mM, and the concentration of fatty acid is in the range of 120 μM to 1 mM.

3. The method according to claim 1, wherein said at least one fatty acid is selected from palmitic acid, oleic acid and linoleic acid.

4. The method according to claim 3, wherein the fatty acid is palmitic acid.

5. The method according to any one of claims 1 to 4, wherein the cells in the cell culture are selected from cells affected in diabetes and obesity status.

6. The method according to any one of claims 1 to 5, wherein the cells in the cell culture are selected from skeletal muscle cells, insulin secreting cells (β-like cells), adipocytes and hepatocytes.

7. A cellular in vitro model system for insulin resistance prepared according to any one of claims 1 to 6.

8. Use of the cellular in vitro model system for insulin resistance according to claim 7 for drug/target related studies.

9. The use according to claim 8, wherein said study is selected from screening of insulin releasing, insulin sensitizing or insulin mimetic compounds, metabolic pathway analysis, differential display analysis, and signaling pathway analysis.

10. The use according to claim 8 or 9, which comprises measuring one or more of glucose uptake rate, glucose oxidation rate and fatty acid oxidation rate in response to action of insulin.

11. A method of screening for insulin releasing, insulin sensitizing or insulin mimetic compounds, which method comprises exposing cells of the in vitro model system according to any one of claims 1 to 7 to (i) at least one compound whose ability to influence the insulin response is sought to be determined, and (ii) insulin, and monitoring said cells for changes in glucose uptake and/or glucose oxidation rate and/or fatty acid oxidation rate.

12. An insulin sensitizing or insulin mimetic compound when identified by the method according to claim 11, or obtained by chemical modification of a compound identified by the method according to claim 11.

13. A device, useful in the use according to claim 10 or in the method according to claim 11, comprising a cell culture flask (1) having an opening (11), and a support member (2) for a carbon dioxide absorbent body, which support member (2) is partially insertable into the culture flask (1) and fixable in the flask opening with the absorbent body extending into the flask.

14. The device according to claim 13, wherein said support is a tubular member (2) provided with a plurality of holes (3) in the tubular wall.

15. The device according to claim 14, wherein the end of said tubular member (2) outside the flask is sealed by a piercable membrane (15).

16. The device according to any one of claims 13 to 15, wherein said absorbent body comprises a filter paper.

17. A method for measuring the rate by which a substrate is oxidized by cells in an in vitro cell culture, comprising the steps of incubating a cell culture in a cultivation flask with a substrate labeled with a radioactive carbon isotope, absorbing carbon dioxide produced by said cells in an absorbent element in contact with the atmosphere within the flask, measuring the amount of radioactive carbon dioxide in said absorbent element, and determining the oxidation rate therefrom, wherein a device according to any one of claims 13 to 16 is used.
